(19) United States
(12) Patent Application Publication
(10) Pub. No.: US 2005/0080156 A1
(43) Pub. Date: Apr. 14, 2005

(54) Tooth filling material

(75) Inventors: Wulf Bramer, Bruchkobel (DE); Ulrich Koops, Rossdorf (DE); Gerhard Dehm, Freigericht (DE)

Correspondence Address: Norris, McLaughlin & Marcus P.A.;30th Floor, 220 East 42nd Street, New York, NY, 10017, US

(73) Assignee: Heraeus Kulzer GmbH & Co., Hanau (DE)

(21) Appl. No.: 10/925,436

(22) Filed: Aug. 25, 2004

(30) Foreign Application Priority Data
Aug. 26, 2003 (DE) 103 39 557.1

(51) Int. Cl. A61F 2/00

(52) U.S. Cl. 523/115

(57) ABSTRACT

A tooth filling material of powder and liquid is described, essentially consisting of a mixture of the following ingredients: A) an organic, self-hardening component and B) an inorganic component that involves a curing reaction with the formation of crystals and expansion, C) water, with the characterizing property that, after mixing, the mixture remains plastically deformable in a processing time of 2 to 5 minutes and then hardens within 10 to 15 minutes.

The invention relates to a tooth filling material.

In dental medicine, more and more people are turning away from amalgam as a tooth filling material, both due to the grayish-black color impression and due to concerns regarding the toxicity of mercury.

The tooth-colored composite fillings form an alternative; such fillings are deposited in layers in the cavity and hardened with light. However, shrinkage during polymerization results in problems where there are openings between tooth and filling material. The so-called margin contraction gap leads to an increased danger of further attack by caries.

Low-shrinkage polymers with ring-opening polymerization (ROMP, DE 199 05 093 A1) or polymers based on siloxane (e.g. DE 41 33 494 C2) have been suggested to circumvent this problem. However, the result of the fact that the dimensions of the usual organic fillers like glasses, glass ceramics or silicate powder do not change is that a slight shrinkage of the material overall remains.

DE 38 13 607A1 describes a composite material filled with metal for dental restoration that has an inorganic, slightly expanding matrix, in particular an inorganic substance that is cable of crystallization. DE 100 21 605 A1 relates to a tooth filling material, especially a root canal filling compound containing a component that expands during hardening due to chemical, physical-chemical or physical effects, especially a system of ammonium salt and base calcium salt. The expanding component here is present in such quantities that the entire material expands during hardening.

Another advantage of the filling materials based on amalgam is the convenient processing. In a freshly mixed state, the material is easily deformable and fits even into complicated interior geometries. It can be pressed into the cavity by exercising pressure (packing). An attempt has already been made to imitate these “handling” characteristics of the amalgam with fine-grained composite compounds (DE 44 43 702 A1/U.S. Pat. No. 5,886,064).

The time of hardening for amalgam is also favorable for the person working with it: after mixing—usually in the capsule in a mixer—the material can be easily handled and packed for 2 to 5 minutes. Then a hardening phase follows that lasts 5 to 10 minutes. The material is then hard enough to allow the patient to go, after a polishing step, with the usual instructions to avoid solid foods for two hours.

The object of the invention is to provide a self-hardening material that comes the closest possible to the known amalgam with respect to three criteria: the characteristic time of hardening, the processing capability and the sealed margins.

Therefore, the present invention involves a tooth filling material that essentially consists of a mixture of the following ingredients:
A) an organic, self-hardening component;
B) an inorganic component that involves a curing reaction with the formation of crystals and expansion, and, if necessary,
C) suitable filler materials, and has the same hardening time curve as silver amalgam, that is it remains plastically deformable in a processing time of 2 to 5 minutes and then hardens within 10 to 15 minutes.

The system is advantageously a two-component system of powder and liquid. The curing and/or polymerization reactions occur when the components are mixed. In this process, it is advantageous to offer the system in capsules, similar to amalgam, which fit on previous mixers.

The organic self-hardening component is a known self-hardening system, e.g. as described in U.S. Pat. No. 4,547,531 or U.S. Pat. No. 5,688,883.

The inorganic component is advantageously a system of powder and water based on cement hardening, e.g. a mixture based on ammonium phosphate and magnesium oxide.

The components are effectively supplied separately as powder and liquids. Generally, the powder component contains filler materials that are still inert that influence the mechanical mixing characteristics. Before use, powder and liquid are mixed to make a paste. This occurs effectively in capsules that are placed in the former amalgam or cement mixer. The mixing movement leads to a combining of the liquid and solid components, advantageously to a paste. The paste can then be removed and handled.

The fact that the hardening behavior is equivalent to that of the previous silver amalgam is important to the invention. This means that the user senses no significant difference with respect to the working method and the time needed for the individual steps. The processing time in which the mass can still be formed is 3 to 5 minutes with the usual silver-tin amalgam. This criterion will also be fulfilled with the material according to the invention. The same is also true for the curing time that lies in the range of 5 to 10 minutes. Ultimately, no adaptation is necessary in comparison to producing amalgam fillings. In this process, the organic two-component mass hardens in a similar amount of time in which the cement hardening of the inorganic component also occurs. The organic component is designed to be self-hardening, i.e., initiators are used, e.g. thermal initiators or redox initiator systems.

In particular, organic peroxides in the form of diacylperoxides, peroxydicarbonates, alkylperesters, dialkylperoxides, perketals, ketone peroxides and alkylhydroperoxides can be considered as thermal initiators. Concrete and preferred examples of thermal initiators are dibenzoylperoxide, t-butylperbenzoate and azobi-isobutyronitrile. The oxidizing part of the redox initiator systems can be e.g. a compound from the group benzoylperoxide, laurylperoxide, benzoin, benzophenone and alpha-diketones. Benzoylperoxide is preferred. The reducing part of the redox initiator systems comes e.g. from the group of tertiary amines. Preferred among them are N,N-dimethyl-para-toluidine and N,N-dimethyl-sym-xylidine.

Preferably, the initiators are contained in the mixture at 0.3 to 1.5 weight-%.

In the selection of filler materials, the consistency of amalgam is the goal. Suitable filler materials include the so-called micro-filling materials whose grain size lies in the nm range, say highly-dispersed silicic acid and so-called macro-filling materials whose grain size lies in the micrometer range, especially granular silicic acid or ground dental glasses. Among the glasses, the preferred are aluminum silicate glasses that can be doped with barium, strontium or rare earths (DE-PS 24 58 380). Ground glasses or quartz with medium particle sizes between about 1 and 10 micrometers are preferred, as well as high-dispersed SiO₂ with average particle sizes between about 10 and 400 nm.

### EXAMPLE

The following has proven to be a suitable mixture:

Inorganic binding system based on ammonium phosphate/magnesium oxide 25%

Inert filler materials 60%.

Organic two-component compounds 15%.

The compound is mixed with an amalgam mixer and put into a cavity.

The processing time until hardening is 4 to 5 minutes.

The hardening time is 10 to 13 minutes.

1. Tooth filling material of powder and water that comprises a mixture of the following ingredients:
A) an organic, self-hardening component;
B) an inorganic component that involves a curing reaction with the formation of crystals and expansion; and
C) water, wherein, after mixing, the mixture remains plastically deformable in a processing time of 2 to 5 minutes and then hardens within 10 to 15 minutes.

2. Tooth filling material according to claim 1, which additionally comprises D) an organic biocompatible solvent.

3. Tooth filling material according to claim 1, which additionally comprises E) suitable filler materials.

4. Tooth filling material according to claim 1, in which the inorganic and/or the organic components expand during hardening.

5. Tooth filling material according to claim 1, in which the organic, self-hardening component is a two-component system of powder and liquid.

6. Tooth filling material according to claim 1, in which the inorganic component is a system of powder and water based on cement hardening.

7. Tooth filling material according to claim 6, in which the powder contains a mixture of ammonium phosphate and magnesium oxide.

8. Tooth filling material according to claim 3, in which the filler materials are a mixture of ground dental glass and silicic acid.

9. Tooth filling material according to claim 8, in which the dental glass is barium aluminum silicate glass.

10. A method of filling a cavity in a tooth, said method comprising the following steps:
A) filling the cavity with the tooth filling material according to claim 1; and
B) allowing the tooth filling material to harden.
